(19) United States
(12) Patent Application Publication
(10) Pub. No.: US 2010/0273193 A1
(43) Pub. Date: Oct. 28, 2010

(54) Triacetone Triperoxide and Diacetone Diperoxide Derivatives, Method for the Preparation and Use Thereof

(75) Inventors: Astrid Walter, Berlin (DE); Rudolf J. Schneider, Berlin (DE); Michael G. Weller, Rangsdorf (DE)

Correspondence Address: Gregory A. Nelson;Novak Druce & Quigg LLP, 525 Okeechobee Blvd, Suite 1500, West Palm Beach, FL, 33401, US

(73) Assignee: BAM Bundesanstalt fur Materialforschung und-pruefung, Berlin (DE)

(21) Appl. No.: 12/767,305

(22) Filed: Apr. 26, 2010

(30) Foreign Application Priority Data
Apr. 27, 2009 (DE) 10 2009 002 652.5

(51) Int. Cl. G01N 33/53; C07D 323/00; C07D 405/12; C07D 323/04; C07K 14/765; C07K 14/77; C12N 9/96; C07H 21/04; C07K 16/44

(52) U.S. Cl. 435/7.92; 549/352; 548/517; 549/367; 530/363; 530/367; 435/188; 536/23.1; 530/388.9; 530/389.8

(57) ABSTRACT

This disclosure is drawn to a triacetone triperoxide derivative in accordance with the general formula (I) and a diacetone diperoxide derivative in accordance with the general formula (II)

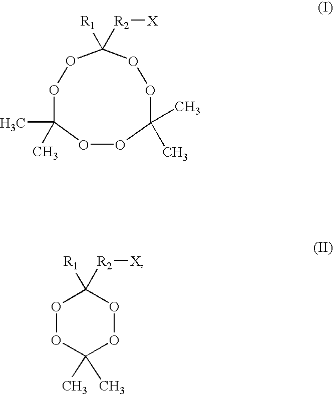

wherein R₁ is a hydrogen residue or an optionally halogenated or perhalogenated C₁ to C₅ alkyl group, R₂ represents a linker molecule, and X represents a reactive or activatable group. A method for the preparation of the TATP or DADP derivatives and the use thereof as antigen for the preparation of TATP- or DADP-specific antibodies or other binders is also disclosed. The antibodies are preferably used in a biosensor for the detection of TATP or DADP in air.

### CROSS-REFERENCE TO RELATED APPLICATIONS

This application claims priority to German Patent Application 102009002652.5, filed Apr. 27, 2009, the contents of which are incorporated herein by reference.

### FIELD OF THE INVENTION

The invention relates to a triacetone triperoxide derivative and a diacetone diperoxide derivative, a method for the preparation as well as the use thereof in the preparation of antibodies and in the preparation of immunosensors for the detection of TATP and/or DADP, especially in air.

### BACKGROUND OF THE INVENTION

Triacetone triperoxide (TATP) or, more correctly, 3,3,6,6,9,9-hexamethyl-1,2,4,5,7,8-hexaoxacyclononane according to IUPAC (CAS No. 17088-37-8), has been synthesized for the first time as early as 1895 from acetone, CH₃COCH₃, and hydrogen peroxide, H₂O₂, in the presence of small amounts of phosphoric acid (R. Wolffenstein “Über die Einwirkung von Wasserstoffsuperoxyd auf Aceton and Mesityloxyd”, Berichte der Deutschen Chemischen Gesellschaft, 28(2), 1895, 2265-2269). The enormous explosiveness of the compound, which can be initiated by impact, friction or heating, has already been observed at that time.

Today, TATP is widely known as an explosive which is used, inter cilia, by suicide assassins. In one aspect, its preferred use in terrorist acts is attributable to the fact that the compound is very easy to prepare from generally available starting materials, such as acetone included in nail polish removers and hydrogen peroxide which can be found in hair bleaches. Having the properties of an initiating explosive, i.e. high sensitivity to shock and impact, a separate, possibly metal-containing and thus easily detectable detonator is not required. In addition, TATP does not contain nitrogen, thereby ruling out a number of methods for the detection thereof, and has a similarly low density as sugar so that it will not be detected by scanners used e.g. in baggage screening.

However, apart from criminal uses, TATP has been repeatedly produced by hobbyists, intentionally or unintentionally, or is occasionally formed as a result of improper disposal of corresponding precursors in chemical waste.

Thus, there is an urgent need for a method or a detection device for the detection of TATP in air, on surfaces of objects, in tissues, etc.

Immunochemical methods are particularly suitable for highly selective and highly sensitive detection of substances, including in particular explosives. The detection is based on strong binding of the analyte, here acting as antigen, to a highly selective antibody and suitably detecting said binding by converting this binding process into an easily detectable signal. For example, the use of the antibodies on a quartz crystal microbalance is well-known (e.g. A. V. Kuznetsov and O. I. Osetrov in “Detection and Disposal of Improvised Explosives” NATO Security through Science Series—B: Physics and Biophysics, H. Schubert and A. Kuznetsov (ed.), Springer-Verlag, 2006, 7-23). A gas sensor having a detection sensitivity for trinitrotoluene (TNT) of 73 ppb, which is equipped with monoclonal TNT antibodies and utilizes the method of surface plasmon resonance (SPR) to detect antibody-antigen binding, has been described in the literature (J. Bowen et al., “Gas-Phase Detection of Trinitrotoluene Utilizing a Solid-Phase Antibody Immobilized on a Gold Film by Means of Surface Plasmon Resonance Spectroscopy”, Applied Spectroscopy 57(8), 2003, 906-914).

The vapor pressure of TATP at room temperature has been determined by gas chromatography to be about 7 Pa and is thus higher than that of TNT by a factor of 10⁴ (J. C. Oxley et al., “Determination of the Vapor Density of Triacetone Triperoxide (TATP) Using a Gas Chromatography Headspace Technique”, Propellants Explosives Pyrotechnics 30(2), 2005, 127-130). Consequently, detection of TATP in air should be possible in principle. To date, however, no immunosensor for TATP is known, nor is a TATP antibody (A. V. Kuznetsov and O. I. Osetrov in “Detection and Disposal of Improvised Explosives” NATO Security through Science Series—B: Physics and Biophysics, H. Schubert and A. Kuznetsov (ed.), Springer-Verlag, 2006, 7-23).

Moreover, diacetone diperoxide (DADP, CAS No. 1073-91-2, IUPAC name: 3,3,6,6-tetramethyl-1,2,4,5-tetraoxane) is well-known, which has similar properties as its trimeric counterpart and is formed from the latter in the presence of particular acids in an extremely slow reaction (F. Dubnikova et al., “Decomposition of triacetone triperoxide is an entropic explosion”, Journal of the American Chemical Society 2005, 127(4), 1146-1159; R. Matyas “Study of TATP: Spontaneous transformation of TATP to DADP” Propellants Explosives Pyrotechnics 33(2), 2008, 89-91).

Antibodies are obtained by immunization of vertebrates, to which end the antigen is usually injected into the latter. In the event of low-molecular weight analytes such as TATP or DADP, however, the substance must be coupled to a (immunogenic) carrier protein in order to be recognized as antigen by the immune system of the animal. However, due to the chemical structure, especially the absence of suitable chemical groups, coupling of TATP or DADP to proteins is not easy to accomplish.

### DETAILED DESCRIPTION OF THE INVENTION

The invention is based on the object of providing derivatives of TATP and DADP, which can be coupled to a protein in order to allow preparation of TATP- or DADP-specific antibodies for use in TATP or DADP sensors. Consequently, another object of the invention is to provide TATP- or DADP-specific antibodies and sensors.

Said object is accomplished by means of a triacetone triperoxide derivative in accordance with the general formula (I),

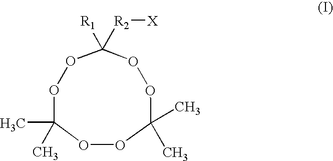

wherein R₁ is a hydrogen residue, H, or an optionally halogenated or perhalogenated C₁ to C₅ alkyl group, R₂ represents a linker molecule, and X represents a reactive or activatable group, which can be coupled in particular covalently or non-covalently (i.e. via physical interactions) to proteins (e.g. Greg. T. Hermanson: “Bioconjugate Techniques”, 2^{nd} revised edition (2008), Academic Press).

Likewise, said object is accomplished by means of a diacetone diperoxide derivative in accordance with the general formula (II) wherein R₁, R₂ and X have the same meaning as in formula (I).

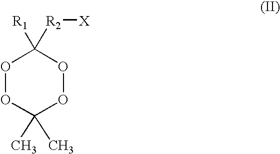

The derivatives according to the invention (which may comprise both enantiomers or the racemate) differ from the actual TATP or DADP target structure in that a methyl residue is substituted with a side chain containing the functional group X. This functional group allows coupling of the derivative to a (immunogenic) carrier protein, in which event the in particular covalent binding can be binding to e.g. a functional group of a side chain on the protein backbone, such as a carboxyl, hydroxyl, amino or thiol group.

Inter alia, one surprising aspect of the derivatives according to the invention is their stability, which is unusual for such peroxides and also allows immunization. Also, this long-term stability allows easy commercial utilization of e.g. conjugates. The substances represent virtually no risk to humans and environment and can be shipped safely in diluted state.

The R₂ linker molecule can be virtually any structure known for example from the specialist literature of immunology (e.g. Greg. T. Hermanson: Bioconjugate Techniques). More specifically, R₂ can be a preferably unbranched, saturated, or mono- or polyunsaturated C₁ to C₂₀ alkyl residue. “Mono- or polyunsaturated alkyl groups” are understood to be hydrocarbon residues having one or more double bonds or triple bonds or combinations thereof in any arrangement. Furthermore, R₂ can be an oligo(ethylene oxide), a peptide, a peptidoid, a nucleic acid, an oligosugar and other synthetic or natural polymers or copolymers and combinations of the above-mentioned groups.

According to a preferred embodiment, R₂ can advantageously be an unbranched, saturated C₁ to C₈ alkyl residue, especially a C₂ to C₇ alkyl residue, preferably a C₅ alkyl residue.

Likewise, functional groups known from the relevant specialist literature (e.g. Greg. T. Hermanson: Bioconjugate Techniques) are possible as couplable (active) group X. More specifically, X can be an amino group, NH₂, a carboxyl group, COOH, an aldehyde group, CHO, a hydroxyl group, OH, a thiol group, SH, an oxirane group (epoxide group), CH(O)CH₂, an aziridine group (ethyleneimine group), CH(NH)CH₂, a hydrazine group, NH—NH₂, a hydrazide group, CONH—NH₂, a hydroxylamine group, ONH₂, an N-hydroxysuccinimide ester or the like. This also includes salts, esters (especially activated esters) and anhydrides of the functional groups X as far as they can result in formation thereof. Non-covalent binding groups are also suitable as couplable group X, such as biotin and derivatives thereof, so-called peptide tags, especially His tags (oligohistidine), and others. The X group can also be a detectable label or may comprise such a label, for instance a dye, especially a fluorescent dye or other, a radioactive label or a stable isotope label.

Furthermore, X is preferably a carboxyl group or an activated ester, a mixed or symmetrical anhydride. Linking such a carboxyl group to a carrier protein can be performed in a particularly simple manner.

In a preferred embodiment of the invention, R₁ is a methyl group.

The triacetone triperoxide derivative according to the invention preferably corresponds to the specific formula (Ia) (6-(3,6,6,9,9-pentamethyl-1,2,4,5,7,8-hexaoxonan-3-yl)hexanoic acid) or an activated ester (e.g. N-hydroxysuccinimide ester, NHS ester) or a mixed anhydride (e.g. prepared using isobutyl chloroformate).

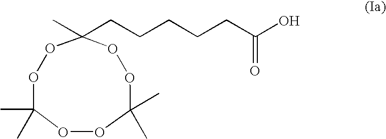

The preferred structure of the diacetone diperoxide derivative according to the invention corresponds to the specific formula (IIa) or an activated ester (e.g. NHS ester) or a mixed anhydride (e.g. prepared using isobutyl chloroformate).

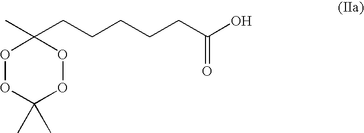

A further aspect of the invention relates to a method for the preparation of the above-described inventive triacetone triperoxide or diacetone diperoxide derivative, wherein acetone, hydrogen peroxide and a compound in accordance with the general formula (III) are reacted,

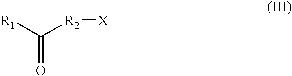

wherein R₁ is a hydrogen residue, H, or an optionally halogenated or perhalogenated C₁ to C₅ alkyl group, R₂ represents a linker molecule, and X represents a functional group which can be coupled covalently or non-covalently.

Preferred embodiments of the R₁ and R₂ residues correspond to those specified in the context with the TATP derivative and DADP derivative according to the invention.

Thus, the preparation of the TATP derivative corresponds to the reaction equation (IV).

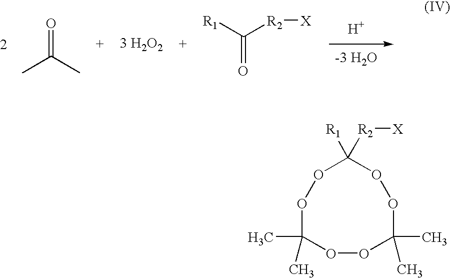

In a preferred fashion, 7-oxooctanoic acid according to the formula (IIIa) is employed as compound in accordance with general formula (III), thereby resulting in the triacetone triperoxide derivative according to formula (Ia).

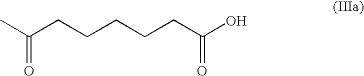

The reaction used herein therefore corresponds to Wolffenstein's approach (see above), with an acetone building block being replaced with the compound according to general formula (III) and in particular with 7-oxooctanoic acid.

The reaction according to the invention is substantially accelerated by the presence of protons, for which reason it is preferably performed in an acidic medium. This can be achieved by adding standard laboratory mineral acids such as sulfuric acid, hydrochloric acid, nitric acid, phosphoric acid, perchloric acid, methanesulfonic acid, formic acid, acetic acid, citric acid or others. Here, sulfuric acid or hydrochloric acid is preferably used. If one of the reactants itself has acidic properties, addition of acid is dispensable, and the reaction can be performed in an autocatalytic fashion.

Particularly good yields and lower amounts of byproducts are obtained when using a batch of acetone, hydrogen peroxide and compound of general formula (III) at a molar ratio of about 1:1:1. In this way it is possible to keep the shares of byproducts at a lower level.

Apart from the water that comes with the hydrogen peroxide, the reaction can be performed without adding solvents because acetone itself is a solvent.

The reaction is preferably performed at room temperature or below.

Another aspect of the present invention relates to the use of the above-described inventive TATP or DADP derivative in covalent or non-covalent linking to proteins (e.g. immunogenic carrier proteins, coating proteins or marker enzymes), DNA, sugars, or labels (e.g. fluorescent dyes, radioisotopes or stable isotopes), inert carriers (e.g. agarose), microparticles or membranes.

In still another aspect the present invention relates to the use of the above-described inventive TATP or DADP derivative as antigen for the preparation of TATP- or DADP-specific binders, especially antibodies, preferably monoclonal antibodies, fragments, derivatives or fusion products thereof, antibody analogs, aptamers, binders based on protein scaffolds, molecularly imprinted systems (MIPs), nanostructured materials, selectively binding surfaces or isotope-labeled compounds. The preparation of antibodies usually proceeds via preferably covalent binding of the derivative to a suitable carrier protein, e.g. ovalbumin, bovine serum albumin or KLH (hemocyanin), and subsequent immunization of vertebrates. To this end, the latter are exposed to the antigen, i.e. a conjugate of carrier protein and TATP or DADP derivative coupled thereto, which is normally effected by multiple injection of the antigens. Following a specific immunization period, the (polyclonal) antibodies having formed can be isolated from the blood of the animal and purified. It is also possible to obtain monoclonal antibodies from animals immunized in this way. The methods used in the preparation of polyclonal and monoclonal antibodies are regarded as belonging to the specialist knowledge of biochemists and molecular biologists and therefore need not be explained in detail herein.

The TATP- or DADP-specific binders thus obtained, especially the antibodies, are preferably used in an affinity sensor (immunosensor) for the detection of triacetone triperoxide (TATP) or diacetone diperoxide (DADP). In addition to the immobilized antibodies, such a sensor includes a detection mechanism which provides a measurable signal when binding between the antibody and TATP or DADP (or a derivative thereof) has taken place. In principle, all detection systems used in well-known immunosensors, especially quartz microbalances, cantilever systems, SPR (surface plasmon resonance) systems and other optical and electrochemical systems, are possible to this end.

The TATP- or DADP-specific binders, especially the antibodies, can also be used in affinity assays (e.g. ELISA) or in affinity-accumulating systems (e.g. immunoaffinity chromatography columns) to detect triacetone triperoxide (TATP) or diacetone diperoxide (DADP).

The antibodies, which antibodies are specific to the triacetone triperoxide and diacetone diperoxide derivatives according to the invention and thus to TATP and DADP themselves, especially monoclonal antibodies, or antibody fragments, as well as a sensor including such antibodies, for the detection of TATP or DADP represent further aspects of the present invention.

Other uses of the antibodies relate to immunoassays (e.g. ELISA) for the detection of TATP or DADP in liquid samples, affinity materials containing immobilized antibodies or haptens for affinity chromatography or affinity accumulation, and immunological quick tests for the detection of TATP or DATP, e.g. in the form of test strips, immunochromatographic systems, lateral-flow tests or similar systems.

Moreover, it is possible to produce other binders specifically interacting with TATP and/or DADP. Such compounds are, for example, antibody fragments (e.g. Fab), recombinant antibodies, DNA or RNA aptamers, binders based on other polymer backbones (protein scaffolds), molecularly imprinted polymers (MIPs), phage display peptides, mimotopes.

Using the derivatives according to the invention, it is also possible to synthesize other TATP or DADP tracers such as enzyme conjugates, fluorescent conjugates, radiotracers, isotope tracers, particle tracers, etc. Another use involves the production of microarrays (immunoarrays or hapten arrays) or bead-based arrays as well as the use of the derivatives for cell labeling in flow cytometry.

Other preferred embodiments of the invention result from the other features specified in the subclaims.

### BRIEF DESCRIPTION OF THE DRAWINGS

The invention will be illustrated in more detail below in the examples and with reference to the associated figures, wherein:

FIG. 1 shows MALDI-TOF mass spectra of a BSA-coupled TATP derivative (solid line) with a coupling density of 11 TATP derivative molecules per BSA molecule and of uncoupled BSA (broken line);

FIG. 2 shows MALDI-TOF mass spectra of a BSA-coupled TATP derivative (solid line) with a coupling density of 15 TATP derivative molecules per BSA molecule and of uncoupled BSA (broken line);

FIG. 3 shows the time profile of the immunization of mice with BSA conjugate of the TATP derivative (a: direct ELISA; b: indirect ELISA); and

FIG. 4 shows the time profile of the immunization of rabbits with BSA conjugate of the TATP derivative (a: direct ELISA; b: indirect ELISA).

### EXAMPLES

### Example 1

### 1.1 Synthesis of a TATP Derivative

The synthesis was carried out in accordance with the reaction equation represented below. 80.71 mg of 7-oxooctanoic acid (98% purity; 0.5 mmol) was weighed and dissolved in 36.76 μl of acetone (0.5 mmol), and 51.05 μl of hydrogen peroxide (30%; 0.5 mmol) was added with mixing. An acidic milieu was adjusted by adding 2.5 μl of sulfuric acid, H₂SO₄ (2 M, 0.005 mmol), at 0° C. (pH value not determined, acetone/acid ratio=1·10⁻²). The batch was allowed to rest for 24 h to about 7 days at room temperature. Thereafter, precipitation with H₂O was effected to obtain a viscous pellet which was taken up in an acetonitrile/water mixture. Purification was effected by means of HPLC-DAD using an acetonitrile/water gradient with TFA.

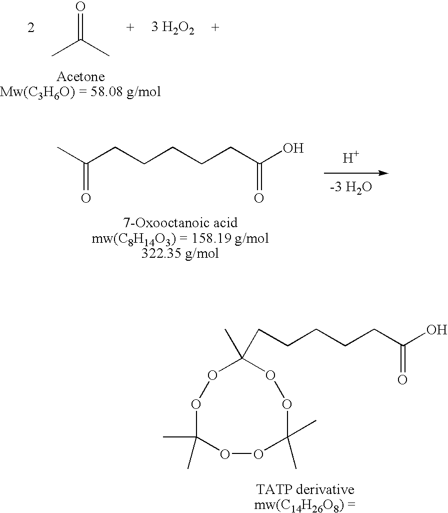

The structure of the purified TATP derivative was verified by means of ¹H-NMR spectroscopy (Broker, 600 MHz) and ¹³C-NMR spectroscopy (Bruker, 150.9 MHz) in MeOD each time. In addition, a high-resolution mass spectrum (ESI negative FT-MS) of the purified TATP derivative was recorded in acetonitrile. The mass of 321.1552 (m/z) that was found deviates from the theoretical (m.w.(C₁₄H₂₅O₈)=321.1555) by less than 0.0001%.

### 1.2 Synthesis of the Activated Ester

The TATP derivative was reacted with N-hydroxysuccinimide (NHS) and dicyclohexylcarbodiimide (DCC) in anhydrous THF or anhydrous DMF at a TATP derivative:NHS:DCC molar ratio of 1:1.2-2:1.2-2 according to the following reaction scheme, strictly maintaining anhydrous conditions. The activated ester of the TATP derivative was obtained in this way.

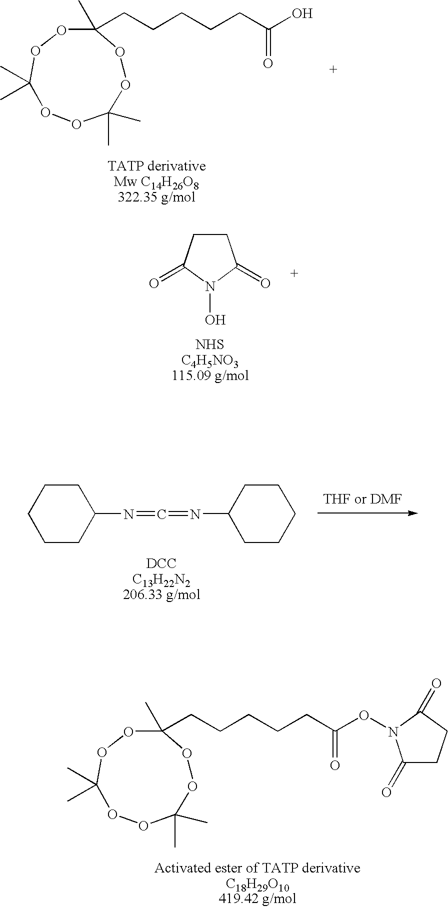

### 1.3 Coupling the Activated Ester to a Protein

The activated ester of the TATP derivative prepared according to the description above was coupled to various proteins in the presence of 0.13 M sodium hydrogen carbonate, NaHCO₃. The proteins bovine serum albumin (BSA, about 66 kDa), horseradish peroxidase (POD, about 44 kDa) and ovalbumin (OVA, about 43 kDa) were used. A covalent peptide bond via a lysine of the respective protein was formed each time. The following reaction scheme shows the coupling reaction with BSA as example.

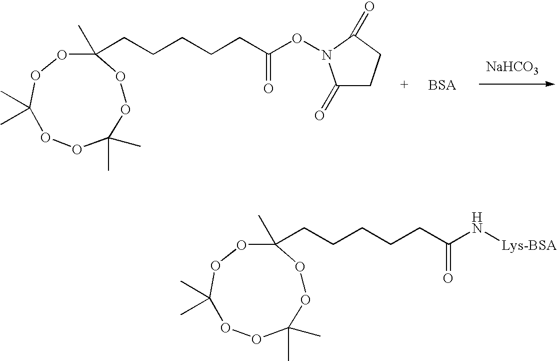

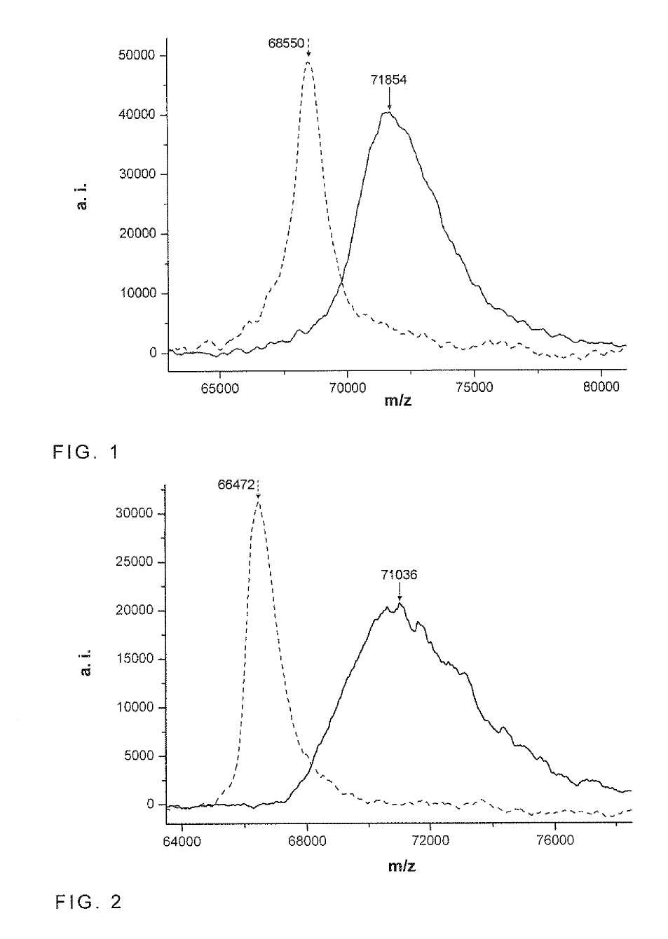

To demonstrate successful coupling of the TATP derivative to the protein, MALDI-TOF MS spectra of two BSA conjugates in comparison with uncoupled BSA were recorded. Desalting of the protein solution prior to measurement was performed on ZebaSpin columns, using sinapic acid as matrix. The spectra were smoothed by taking a sliding average (100 points) and subjected to base line correction. No mass calibration of the MALDI-TOF mass spectrometer was made. The mass spectra are shown in FIG. 1 wherein the broken line shows the spectrum of uncoupled BSA (educt) and the solid line that of the BSA conjugate (=immunogen) for the immunization of mice, which has a mean coupling density of 11 haptens (TATP derivative molecules) per BSA molecule. FIG. 2 illustrates analogous mass spectra wherein the mean coupling density is 15 haptens (TATP derivative molecules) per BSA molecule.

Coupling with horseradish peroxidase (POD) and ovalbumin (OVA) achieved mean coupling densities of 2 and 4 haptens (TATP derivative molecules), respectively, per protein molecule (not shown).

### 1.4. TATP Antibody Production in Mice

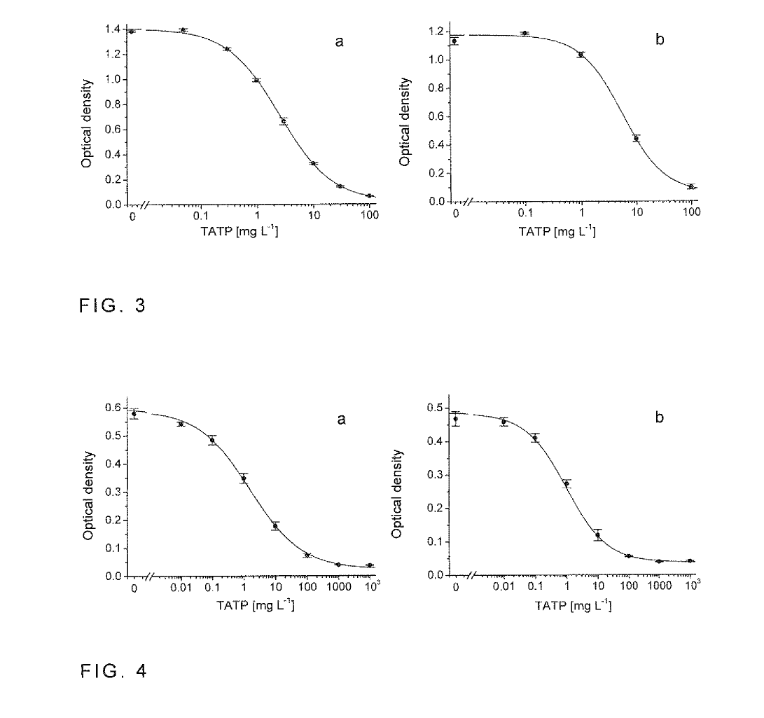

Three BALB/c mice were immunized using the BSA conjugate shown in 1.3, to which end 50 μg of immunogen was injected every 4 weeks. Initial immunization was effected using complete Freund's adjuvant, and subsequent immunizations were performed using incomplete Freund's adjuvant. Using the serums collected from the animals 7 days after each immunization, it was possible to monitor the course of immunization by means of ELISA (enzyme-linked immunosorbent assay), resulting in the sigmoidal TATP calibration curves shown in FIG. 3 (error bars:standard deviation). Curve a shows a direct ELISA with 1:10,000 diluted mouse serum and 1:10,000 diluted TATP-peroxidase tracer (TATP derivative-POD conjugate). The point of inflexion is at 2.5 mg/l; the mean antibody affinity is about 5.7 μM, and the detection limit is around 65 μg/l (n=4). Curve b shows an indirect ELISA with 1:10,000 diluted mouse serum and 1:5,000 diluted anti-mouse peroxidase tracer. The point of inflexion is at 5.6 mg/l; the mean antibody affinity is about 13 μM, and the detection limit is around 870 μg/l (n=3).

### 1.4 TATP Antibody Production in Rabbits

Two rabbits were immunized using the BSA conjugate shown in 1.3, to which end 50 μg of immunogen was injected every 4 weeks. Immunization was effected using an adjuvant. Using the serums collected from the animals 7 days after each immunization, it was possible to monitor the course of immunization by means of ELISA, resulting in the sigmoidal TATP calibration curves shown in FIG. 4 (error bars:standard deviation). Curve a shows a direct ELISA with 1:120,000 diluted rabbit serum and 1:30,000 diluted TATP-peroxidase tracer (TATP derivative-POD conjugate). The point of inflexion is at 1.6 μg/l; the mean antibody affinity is about 5.7 nM, and the detection limit is around 92 ng/l (n=3). Curve b shows an indirect ELISA with 1:120,000 diluted rabbit serum and 1:30,000 diluted TATP-peroxidase tracer (TATP derivative-POD conjugate). The point of inflexion is at 0.98 μg/l; the mean antibody affinity is about 3.5 nM, and the detection limit is around 170 ng/l (n=3).

### 1.5 Determination of Cross Reactions (Selectivity)

Using the serums of the two rabbits from Example 1.4, the cross-reactivities relative to TATP for the individual reactants of the TATP derivative synthesis (7-oxooctanoic acid, H₂O₂ and acetone) were determined. The cross-reactivities were determined using the quotients of the points of inflexion (in μg/l) of the sigmoidal calibration curves. The following table shows that the polyclonal antibodies are highly selective. The results were obtained both in direct and indirect ELISAs.

TABLE

| | Rabbit 1 | Rabbit 2 |
|---|---|---|
| TATP | 100% | 100% |
| 7-Oxooctanoic acid | <0.1% | <0.1% |
| H₂O₂ | <0.1% | <0.1% |
| Acetone | <0.1% | <0.1% |

### Example 2

### 2. Synthesis of a DADP Derivative

The synthesis was carried out in analogy to the TATP synthesis as in Dubnikova et al. (Decomposition of triacetone triperoxide is an entropic explosion. Journal of the American Chemical Society 2005, 127(4), 1148).

We claim:

1. A triacetone triperoxide derivative in accordance with the general formula (I) or a diacetone diperoxide derivative in accordance with the general formula (II), 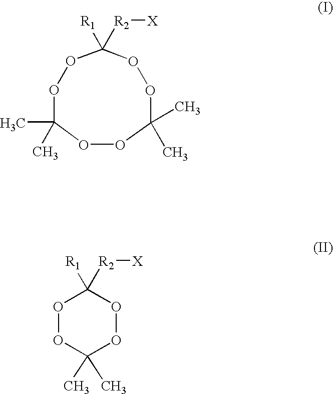
wherein R₁ is a hydrogen residue or an optionally halogenated or perhalogenated C₁ to C₅ alkyl group, R₂ represents a linker molecule, and X represents a reactive or reactivatable group.

2. The TATP or DADP derivative according to claim 1, wherein R₂ is a saturated or mono- or polyunsaturated C₁ to C₂₀ alkyl residue, an oligo(ethylene oxide), a peptide, a peptidoid, a nucleic acid, an oligosugar, a synthetic or natural polymer or copolymer or a combinations of the above-mentioned groups.

3. The TATP or DADP derivative according to claim 2, wherein R₂ is an unbranched saturated C₁ to C₈ alkyl residue, especially a C₂ to C₇ alkyl residue, preferably a C₅ alkyl residue.

4. The TATP or DADP derivative according to claim 1, wherein X represents an amino group, a carboxyl group, an aldehyde group, a hydroxyl group, a thiol group, an oxirane group, an aziridine group, a hydrazine group, a hydroxylamine group, a hydrazide group, an N-hydroxysuccinimide ester or a salt, ester or anhydride thereof, or corresponds to a group that can be non-covalently coupled to proteins, particularly biotin or a derivative thereof, or a peptide tag, especially oligohistidine.

5. The TATP or DADP derivative according to claim 1, wherein X is or comprises a detectable label, especially a dye, a radioactive label or a stable isotope label.

6. The TATP or DADP derivative according to claim 1, wherein R₁ is a methyl group.

7. The TATP or DADP derivative according to claim 1, said triacetone triperoxide derivative being 6-(3,6,6,9,9-pentamethyl-1,2,4,5,7,8-hexaoxonan-3-yl)hexanoic acid in accordance with the formula (Ia) or an activated ester, an anhydride or acid halide thereof

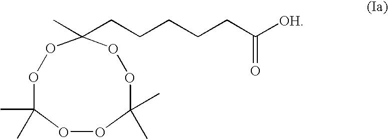

8. A method for the preparation of a triacetone triperoxide or diacetone diperoxide derivative according to claim 1, comprising reacting (i) acetone, an acetone derivative or a precursor releasing acetone with (ii) hydrogen peroxide, a hydrogen peroxide derivative or a precursor releasing hydrogen peroxide and a (iii) compound in accordance with the general formula (III), 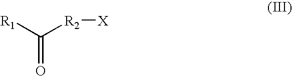
wherein R₁, R₂ and X have the meanings specified above.

9. The method according to claim 8, wherein the compound according to general formula (III) is 7-oxooctanoic acid according to formula (IIIa)

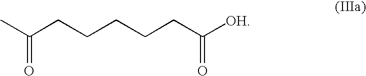

10. The method according to claim 8, wherein the reaction is carried out in the presence of an acid, or autocatalytically using a reactant having acidic properties.

11. The method according to claim 8, wherein acetone, hydrogen peroxide and the compound in accordance with the general formula (III) are used at a molar ratio of about 1:1:1.

12. A method of producing a TATP or DADP complex, comprising:
coupling, either covalently or non-covalently, a TATP or DADP derivative according to claim 1 to a compound selected from the group consisting of a protein, DNA and a label.

13. A method of forming a TATP- or DADP-specific binder comprising:
introducing a compound comprising a TATP or DADP derivative according to claim 1 coupled to a carrier protein into an animal.

14. The method according to claim 13, wherein the TATP- or DADP-specific binder is selected from the group consisting of monoclonal or polyclonal antibodies, fragments, derivatives or fusion products thereof, antibody analogs, aptamers, binders based on protein scaffolds, molecularly imprinted systems, nanostructured materials, selectively binding surfaces and isotope-labeled compounds.

15. A method of detecting triacetone triperoxide (TATP) or diacetone diperoxide (DADP), comprising:
providing an affinity sensor, an affinity assay or an affinity-accumulating system comprising a TATP- or DADP-specific binder produced by the method of claim 13; and
detecting TATP or DADP using said affinity sensor, said affinity assay or said affinity-accumulating system.

16. An antibody or antibody fragment against a triacetone triperoxide or diacetone diperoxide derivative according to claim 1.

17. A sensor for the detection of triacetone triperoxide or diacetone diperoxide, comprising a TATP- or DADP-specific binder produced by the method of claim 13.
